(19) United States
(12) Patent Application Publication
(10) Pub. No.: US 2025/0351843 A1
(43) Pub. Date: Nov. 20, 2025

(54) Method for Producing Low-toxin, Low-viscosity and Enhanced-quality Corn Steep Liquor Protein Powder with Special Flavor

(71) Applicant: Jiangnan University, Wuxi (CN)

(72) Inventors: Xiulan SUN, Wuxi (CN); Jian JI, Wuxi (CN); Jiadi SUN, Wuxi (CN); Yang YANG, Wuxi (CN); Lina SHENG, Wuxi (CN); Heyang HUANG, Wuxi (CN); Song GAO, Wuxi (CN); Yinzhi ZHANG, Wuxi (CN)

(21) Appl. No.: 19/280,113

(22) Filed: Jul. 25, 2025

(30) Foreign Application Priority Data
Nov. 1, 2024 (CN) 2024115524535

(51) Int. Cl. A23K 10/12; A23J 3/14; A23K 10/37; A23K 20/147; C12N 1/14; C12R 1/685

(52) U.S. Cl. CPC A23K 10/12; A23J 3/14; A23K 10/37; A23K 20/147; C12N 1/145; C12R 2001/685

(57) ABSTRACT

The disclosure discloses a production method for low-toxin, low-viscosity and enhanced-quality corn steep liquor protein powder with a special flavor, belonging to the field of biological technologies. By inoculating a corresponding proportion of 10⁶ CFU/mL Aspergillus niger spore suspension into corn steep liquor and cultivating it at a certain rotation speed and temperature, the viscosity of the corn steep liquor effectively reduced, and the aflatoxins contamination is significantly reduced. The degradation rate of AFB1, AFB2, AFG1, AFG2 reach 52.14%, 80.21%, 86.31%, 80.22%. After fermentation, the protein content and total amino acid content of the corn steep liquor are increased after drying, and it can be dried into corn steep liquor powder. The disclosure provides a technical reference for the safe and efficient utilization of the corn steep liquor, greatly reduces the transportation cost of the corn steep liquor, extends the product storage period, and improves the convenience in use.

### TECHNICAL FIELD

The disclosure relates to a method for producing low-toxin, low-viscosity and enhanced-quality corn steep liquor protein powder with a special flavor, belonging to the field of microbial technologies.

### BACKGROUND

The process of deep processing of corn mainly includes the following steps. Firstly, the corn is soaked in sulfurous acid water to soften the corn tissue and prepare for subsequent separation. Subsequently, various valuable components, including corn soaking solution, corn germs, corn husks, and pure corn starch, are isolated from the corn. Corn Steep Liquor (CSL) is the main byproduct of deep processing of corn, rich in various chemical nutrients. Concentrated corn steep liquor is obtained by using corn soaking solution to produce phytic acid, inositol and other products, collecting the liquid generated in the production process, and performing combination and concentration. The protein content of this part is 16-30%, the amino acid content is 8-12%, the reducing sugar content is 5-7%, the vitamin content is 0.7-1 mg/L, the total acid content is 8-13%, the lactic acid content is 7-12%, the phosphorus content is 4-4.5%, and the potassium content is 2-2.5%. It is a preferred raw material for animal feed.

In the storage process of corn, mold growth may occur due to the influence of external environment and storage conditions, especially when contaminated by toxic fungi such as Aspergillus flavus. Common aflatoxins include AFB1, AFB2, AFG1, and AFG2. In the existing technology, the degradation of aflatoxins is mostly concentrated in AFB1. For example, Chinese patent CN113373060A discloses Aspergillus niger capable of degrading AFB1. However, aflatoxins AFB1, AFB2, AFG1, and AFG2 often coexist in food and feed, and the degradation of a single toxin cannot completely eliminate the harm of aflatoxins. Therefore, strains that can simultaneously degrade all four types of aflatoxins not only improve degradation efficiency, but also significantly reduce food safety risks, providing a more effective solution for comprehensive prevention and control of aflatoxin contamination.

In addition, due to the high concentration of polysaccharides and lactic acid substances in concentrated corn steep liquor, the concentrated corn steep liquor has a strong viscosity. This high viscosity not only increases the difficulty of the concentration process, but also poses significant challenges to the drying process. Specifically, high-viscosity substances have poor performance in flow and heat transfer, which slows down the flow of the concentrated corn steep liquor in the evaporator and affects the evaporation efficiency. In addition, materials with high viscosity are more likely to form clumps in the drying process, which not only reduces the drying efficiency but may also lead to a decrease in the product quality.

Based on the above statement, providing a production method for corn steep liquor powder that can simultaneously degrade the four types of aflatoxins, reduce the viscosity of powder, and improve the drying efficiency of corn steep liquor has a significant economic benefit. However, there is currently no relevant research in the existing technology.

### SUMMARY

### Technical Solution

In view of this, an embodiment of the disclosure provides a method for preparing flavored, detoxified and viscosity-reduced corn steep liquor powder, so as to solve the problem of ineffective detoxification of toxins AFB1, AFB2, AFG1, and AFG2 in corn steep liquor in the existing technology, consume residual sugars and lactic acid in the corn steep liquor, reduce the viscosity of the corn steep liquor, solve the problems that the viscosity of the corn steep liquor is high and it is difficult to dry, and improve the flavor of the corn steep liquor through fermenting strains.

In order to achieve the above objectives, embodiments of the disclosure provide the following technical solutions.

The disclosure firstly provides a method for preparing corn steep liquor powder with reduced content of aflatoxins. The method includes: using corn steep liquor as a fermentation raw material and performing fermentation by using A. niger. The aflatoxins include at least two of aflatoxins AFB1, AFB2, AFG1, and AFG2.

In an embodiment, the A. niger includes A. niger CCTCC NO: M 2013703.

In an embodiment, the aflatoxins include aflatoxins AFB1, AFB2, AFG1, and AFG2.

In an embodiment, the method includes the following steps:
(1) preparation of seed culture solution: preparing A. niger spore suspension, inoculating the A. niger spore suspension into a culture medium, and performing culture to obtain seed culture solution;
(2) fermentation: inoculating the seed culture solution obtained in step (1) into corn steep liquor, and performing fermentation to obtain a fermentation product; and
(3) preparation of corn steep liquor powder: collecting the fermentation product obtained in step (2) and performing drying to obtain corn steep liquor powder.

In an embodiment, a concentration of A. niger spores in the A. niger spore suspension is not less than 1×10⁶ CFU/mL.

In an embodiment, step (1) specifically includes inoculating the A. niger spore suspension into the culture medium according to an inoculation rate of 1%-3%, and performing culture at 25° C.-35° C. for at least 24 h to obtain the seed culture;
step (2) specifically includes inoculating the seed culture solution obtained in step (1) into corn steep liquor according to an inoculation rate of 5%-25%, and performing fermentation at 25° C.-32° C. for 48-72 h to obtain a fermentation product; and
step (3) specifically includes drying the fermentation product obtained in step (2) at 90° C.-105° C. for 24 h-48 h, and performing crushing.

In an embodiment, the corn steep liquor in step (2) is subjected to sterilization treatment.

In an embodiment, the method includes the following steps:
(1) preparation of seed culture solution: inoculating the A. niger spore suspension into a culture medium according an inoculation rate of 2%, and performing culture at 30° C. for at least 16 h to obtain seed culture solution;
(2) fermentation: inoculating the seed culture solution obtained in step (1) into corn steep liquor according to an inoculation rate of 15%, and performing fermentation at 30° C. for 60 h to obtain a fermentation product; and
(3) preparation of corn steep liquor powder: drying the fermentation product obtained in step (2) at 100° C. for 32 h, and performing crushing.

The disclosure further provides corn steep liquor powder prepared by adopting the method described above.

The disclosure further provides application of A. niger CCTCC NO: M 2013703 to degradation of aflatoxins. The aflatoxins include at least two of aflatoxins AFB1, AFB2, AFG1, and AFG2.

In an embodiment, the aflatoxins include aflatoxins AFB1, AFB2, AFG1, and AFG2.

The disclosure further provides application of the method or the corn steep liquor powder to preparation of feed.

The disclosure has the following beneficial effects:

1. The method in the disclosure can effectively remove AFB1, AFB2, AFG1, and AFG2 from the corn steep liquor, the detoxification steps are simple, the detoxification effect is good, and the removal rates respectively reach 52.14%, 80.21%, 86.31%, and 80.22%.

2. The fermenting strains used in the disclosure can alter the amino acid composition in the corn steep liquor, thereby improving the flavor of the corn steep liquor and making it have better palatability.

3. The viscosity of the corn steep liquor obtained in the disclosure is significantly reduced compared to the viscosity before treatment, so it can be directly used for producing the corn steep liquor powder by using a drying device, thereby greatly reducing the transportation cost, extending the product storage life, and improving the convenience in use.

4. The treatment method described in the disclosure only requires the addition of a small amount of equipment such as a corn steep liquor treatment tank and a supporting mixer, thereby making the process simple and the cost low.

### BRIEF DESCRIPTION OF FIGURES

In order to describe the embodiments of the disclosure or the technical solutions in the existing technology more clearly, the drawings required for the description of the embodiment or the existing technology will be briefly introduced. Clearly, the drawings described below are only exemplary. For those skilled in the art, other drawings may be derived from the provided drawings without contributing any inventive labor.

FIG. 1A shows a state of corn steep liquor after concentration and drying before fermentation.

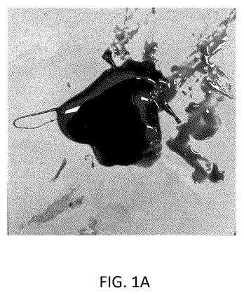

FIG. 1B shows a state of corn steep liquor after concentration and drying after fermentation.

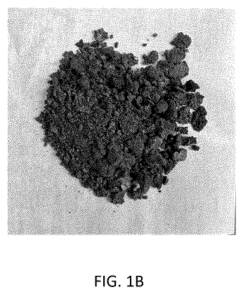

### DETAILED DESCRIPTION

Embodiments of the disclosure will be described below through specific examples. Those skilled in the art may easily understand other advantages and effects of the disclosure from the content disclosed in the description. Clearly, the described examples are a part of the embodiments of the disclosure, not all of them. Based on the examples of the disclosure, all other examples obtained by those skilled in the art without contributing any inventive labor are within the scope of protection of the disclosure.

In the examples of the disclosure, corn steep liquor is concentrated solution obtained after soaking corn particles in sulfur dioxide solution in reverse flow, pumping out soaking solution containing 6-7% of dry matters, and then concentrating the solution to concentrated solution containing a certain number of dry matters through triple-effect vacuum evaporation.

In the examples of the disclosure, the so-called “dry matters” are substances remained after corn steep liquor is dried.

In the examples of the disclosure, A. niger refers to A. niger CCTCC NO: M 2013703, which is disclosed in Chinese patent CN103937681B.

### Example 1

A method for producing low-toxin and low-viscosity corn steep liquor powder with a special flavor included the following steps:

### S1: Activation of A. Niger Strains and Preparation of Seed Culture Solution

A. niger was taken out from −80° C., revived, and inoculated into a PDA culture medium and cultured at 28° C. for 5 days. Spores were washed off with sterile physiological saline containing Tween 80. Spore concentration was adjusted to 1×10⁶ CFU/mL to obtain A. niger spore suspension. The PDB culture medium was pumped into a 5 L fermentation tank and sterilized. After sterilization and cooling, the temperature was increased to 30° C. and maintained. At a rotation speed of 500 r/min, 1% A. niger spore suspension was inoculated and cultured for 24 h to obtain seed culture solution.

### S2: Inoculation and Fermentation of Corn Steep Liquor

The corn steep liquor was pumped into a 10 L fermentation tank and sterilized. After sterilization and cooling, the temperature was increased to 25° C. and maintained. At a rotation speed of 800 r/min and an aeration rate of 1 vvm, 5% A. niger seed culture solution obtained in S1 was inoculated and cultured for 72 h to obtain fermented corn steep liquor with low toxicity, low viscosity, and a special flavor.

The corn steep liquor with low toxicity, low viscosity, and special flavor obtained is S2 was evenly distributed in a stainless steel tray. Drying was carried out in a hot air oven at 90° C. for 48 h. The material obtained after drying was corn steep liquor powder, which presented a non-sticky block shape.

### Example 2

A method for producing low-toxin and low-viscosity corn steep liquor powder with a special flavor included the following steps:

### S1: Activation of A. Niger Strains and Preparation of Seed Culture Solution

A. niger was taken out from −80° C., revived, and inoculated into a PDA culture medium and cultured at 28° C. for 5 days. Spores were washed off with sterile physiological saline containing Tween 80. Spore concentration was adjusted to 1×10⁶ CFU/mL to obtain A. niger spore suspension. The PDB culture medium was pumped into a 5 L fermentation tank and sterilized. After sterilization and cooling, the temperature was increased to 30° C. and maintained. At a rotation speed of 500 r/min, 1.5% A. niger spore suspension was inoculated and cultured for 20 h to obtain seed culture solution.

### S2: Inoculation and Fermentation of Corn Steep Liquor

The corn steep liquor was pumped into a 10 L fermentation tank and sterilized. After sterilization and cooling, the temperature was increased to 28° C. and maintained. At a rotation speed of 700 r/min and an aeration rate of 1 vvm, 10% A. niger seed culture solution obtained in S1 was inoculated and cultured for 66 h to obtain fermented corn steep liquor with low toxicity, low viscosity, and a special flavor.

The corn steep liquor with low toxicity, low viscosity, and special flavor obtained is S2 was evenly distributed in a stainless steel tray. Drying was carried out in a hot air oven at 95° C. for 36 h. A material obtained after drying was corn steep liquor powder, which presented a non-sticky block shape.

### Example 3

A method for producing low-toxin and low-viscosity corn steep liquor powder with a special flavor included the following steps:

### S1: Activation of A. Niger Strains and Preparation of Seed Culture Solution

A. niger was taken out from −80° C., revived, and inoculated into a PDA culture medium and cultured at 28° C. for 5 days. Spores were washed off with sterile physiological saline containing Tween 80. Spore concentration was adjusted to 1×10⁶ CFU/mL to obtain A. niger spore suspension. The PDB culture medium was pumped into a 5 L fermentation tank and sterilized. After sterilization and cooling, the temperature was increased to 30° C. and maintained. At a rotation speed of 500 r/min, 2% A. niger spore suspension was inoculated and cultured for 16 h to obtain seed culture solution.

### S2: Inoculation and Fermentation of Corn Steep Liquor

The corn steep liquor was pumped into a 10 L fermentation tank and sterilized. After sterilization and cooling, the temperature was increased to 30° C. and maintained. At a rotation speed of 600 r/min and an aeration rate of 1 vvm, 15% A. niger seed culture solution obtained in S1 was inoculated and cultured for 60 h to obtain fermented corn steep liquor with low toxicity, low viscosity, and a special flavor.

The corn steep liquor with low toxicity, low viscosity, and special flavor obtained is S2 was evenly distributed in a stainless steel tray. Drying was carried out in a hot air oven at 100° C. for 32 h. A material obtained after drying presented a non-sticky block shape.

### Example 4

A method for producing low-toxin and low-viscosity corn steep liquor powder with a special flavor included the following steps:

### S1: Activation of A. Niger Strains and Preparation of Seed Culture Solution

A. niger was taken out from −80° C., revived, and inoculated into a PDA culture medium and cultured at 28° C. for 5 days. Spores were washed off with sterile physiological saline containing Tween 80. Spore concentration was adjusted to 1×10⁶ CFU/mL to obtain A. niger spore suspension. The PDB culture medium was pumped into a 5 L fermentation tank and sterilized. After sterilization and cooling, the temperature was increased to 30° C. and maintained. At a rotation speed of 500 r/min, 2.5% A. niger spore suspension was inoculated and cultured for 12 h to obtain seed culture solution.

### S2: Inoculation and Fermentation of Corn Steep Liquor

The corn steep liquor was pumped into a 10 L fermentation tank and sterilized. After sterilization and cooling, the temperature was increased to 32° C. and maintained. At a rotation speed of 500 r/min and an aeration rate of 1 vvm, 20% A. niger seed culture solution obtained in S1 was inoculated and cultured for 54 h to obtain fermented corn steep liquor with low toxicity, low viscosity, and a special flavor.

The corn steep liquor with low toxicity, low viscosity, and special flavor obtained is S2 was evenly distributed in a stainless steel tray. Drying was carried out in a hot air oven at 105° C. for 30 h. A material obtained after drying was corn steep liquor powder, which presented a non-sticky block shape.

### Example 5

A method for producing low-toxin and low-viscosity corn steep liquor powder with a special flavor included the following steps:

### S1: Activation of A. Niger Strains and Preparation of Seed Culture Solution

A. niger was taken out from −80° C., revived, and inoculated into a PDA culture medium and cultured at 28° C. for 5 days. Spores were washed off with sterile physiological saline containing Tween 80. Spore concentration was adjusted to 1×10⁶ CFU/mL to obtain A. niger spore suspension. The PDB culture medium was pumped into a 5 L fermentation tank and sterilized. After sterilization and cooling, the temperature was increased to 30° C. and maintained. At a rotation speed of 500 r/min, 3% A. niger spore suspension was inoculated and cultured for 12 h to obtain seed culture solution.

### S2: Inoculation and Fermentation of Corn Steep Liquor

The corn steep liquor was pumped into a 10 L fermentation tank and sterilized. After sterilization and cooling, the temperature was increased to 25° C. and maintained. At a rotation speed of 500 r/min and an aeration rate of 1 vvm, 25% A. niger seed culture solution obtained in S1 was inoculated and cultured for 48 h to obtain fermented corn steep liquor with low toxicity, low viscosity, and a special flavor.

The corn steep liquor with low toxicity, low viscosity, and special flavor obtained is S2 was evenly distributed in a stainless steel tray. Drying was carried out in a hot air oven at 105° C. for 24 h. A material obtained after drying was corn steep liquor powder, which presented a non-sticky block shape.

### Example 6

A method for producing low-toxin and low-viscosity corn steep liquor powder with a special flavor included the following steps:

### S1: Activation of A. Niger Strains and Preparation of Seed Culture Solution

A. niger was taken out from −80° C., revived, and inoculated into a PDA culture medium and cultured at 28° C. for 5 days. Spores were washed off with sterile physiological saline containing Tween 80. Spore concentration was adjusted to 1×10⁶ CFU/mL to obtain A. niger spore suspension. The PDB culture medium was pumped into a 5 L fermentation tank and sterilized. After sterilization and cooling, the temperature was increased to 30° C. and maintained. At a rotation speed of 500 r/min, 2% A. niger spore suspension was inoculated and cultured for 24 h to obtain seed culture solution.

### S2: Inoculation and Fermentation of Corn Steep Liquor

The corn steep liquor was pumped into a 10 L fermentation tank and sterilized. After sterilization and cooling, the temperature was increased to 28° C. and maintained. At a rotation speed of 900 r/min and an aeration rate of 1 vvm, 10% A. niger seed culture solution obtained in S1 was inoculated and cultured for 60 h to obtain fermented corn steep liquor with low toxicity, low viscosity, and a special flavor.

The corn steep liquor with low toxicity, low viscosity, and special flavor obtained is S2 was evenly distributed in a stainless steel tray. Drying was carried out in a hot air oven at 100° C. for 32 h. A material obtained after drying was corn steep liquor powder, which presented a non-sticky block shape.

### Example 7

A method for producing low-toxin and low-viscosity corn steep liquor powder with a special flavor included the following steps:

### S1: Activation of A. Niger Strains and Preparation of Seed Culture Solution

A. niger was taken out from −80° C., revived, and inoculated into a PDA culture medium and cultured at 28° C. for 5 days. Spores were washed off with sterile physiological saline containing Tween 80. Spore concentration was adjusted to 1×10⁶ CFU/mL to obtain A. niger spore suspension. The PDB culture medium was pumped into a 5 L fermentation tank and sterilized. After sterilization and cooling, the temperature was increased to 30° C. and maintained. At a rotation speed of 500 r/min, 2% A. niger spore suspension was inoculated and cultured for 24 h to obtain seed culture solution.

### S2: Inoculation and Fermentation of Corn Steep Liquor

The corn steep liquor was pumped into a 10 L fermentation tank and sterilized. After sterilization and cooling, the temperature was increased to 28° C. and maintained. At a rotation speed of 300 r/min and an aeration rate of 1 vvm, 10% A. niger seed culture solution obtained in S1 was inoculated and cultured for 60 h to obtain fermented corn steep liquor with low toxicity, low viscosity, and a special flavor.

The corn steep liquor with low toxicity, low viscosity, and special flavor obtained is S2 was evenly distributed in a stainless steel tray. Drying was carried out in a hot air oven at 100° C. for 32 h. A material obtained after drying was corn steep liquor powder, which presented a non-sticky block shape.

The removal effects of aflatoxins of method examples 1-7 were detected according to the following steps.

Fermented samples were collected and dried. Then, each portion of corn steep liquor powder was introduced into a centrifuge tube. Acetonitrile-water solution (70+30) was added for extraction. A fermentation dish was rinsed. All obtained solution was introduced into the centrifuge tube to avoid the influence of uneven distribution of toxins in the meal on the experimental results. After uniform mixing, ultrasonic treatment was performed for 10 min, oscillation was performed for 30 min, and centrifugation was performed at 8000 r/min for 10 min. Supernatant obtained after filtration was taken to pass through a solid-phase purification column. Liquid was collected, and then freeze concentration was performed. After redissolving a filter membrane with methanol-water solution (50+50), LC-MS/MS detection was performed. Chromatographic conditions: chromatographic column: BEH C 18 column (2.1 mm×100 mm, 1.7 μm); mobile phase A: acetonitrile; mobile phase B: ammonium formate solution; column temperature: 45° C.; flow rate: 0.3 mL/min; and sample size: 5 μL. For elution conditions, see Table 1.

TABLE 1
Gradient elution table

| | | |
|---|---|---|
| Time | Flow rate | Mobile phase (%) |

| (min) | (mL/min) | A | B |
|---|---|---|---|
| 0.0 | 0.3 | 10 | 90 |
| 0.1 | 0.3 | 10 | 90 |
| 6.0 | 0.3 | 60 | 40 |
| 6.1 | 0.3 | 10 | 90 |
| 7.1 | 0.3 | 10 | 90 |

The residual amounts of four toxins in the samples after removing aflatoxins in examples 1 to 7 were detected, and the corresponding removal rates of the four aflatoxins were calculated. For results, see Table 2.

TABLE 2
Degradation rates of AFB1, AFB2, AFG1, and AFG2

| | Removal | Removal | Removal | Removal |
|---|---|---|---|---|
| Item of | rate of | rate of | rate of | rate of |
| detection | AFB1 | AFB2 | AFG1 | AFG2 |
| Example 1 | 45.32% | 78.97% | 85.27% | 76.39% |
| Example 2 | 47.58% | 77.78% | 85.52% | 79.63% |
| Example 3 | 52.14% | 80.21% | 86.31% | 80.22% |
| Example 4 | 49.26% | 74.65% | 85.16% | 79.96% |
| Example 5 | 42.38% | 69.68% | 82.78% | 76.35% |
| Example 6 | 51.36% | 78.35% | 83.31% | 82.57% |
| Example 7 | 42.19% | 62.35% | 79.23% | 73.52% |

Contents of protein, amino acid, and dry matters were determined according to standards GB/T 6432-2018, GB/T 18246-2000, and GB/T 22428.4-2008 respectively. The residual sugar concentration was determined by using Sieman M-100. For results, see Table 3.

TABLE 3
Changes in total protein, total amino acid, total dry matter,
and residual sugar before and after fermentation

| Item of | Total | Total amino | Total dry | Residual |
|---|---|---|---|---|
| detection | protein | acid | matter | sugar |
| Before | 49.36% | 39.61% | 6.11% | 1.15 g/L |
| fermentation | | | | |
| Example 1 | 53.52% | 53.17% | 5.16% | 0.14 g/L |
| Example 2 | 55.64% | 54.96% | 5.03% | 0.13 g/L |
| Example 3 | 54.97% | 51.35% | 5.38% | 0.17 g/L |
| Example 4 | 53.62% | 51.62% | 5.23% | 0.12 g/L |
| Example 5 | 51.72% | 47.92% | 5.66% | 0.21 g/L |
| Example 6 | 54.68% | 52.32% | 5.98% | 0.09 g/L |
| Example 7 | 50.69% | 47.29% | 5.37% | 0.19 g/L |

Volatile compounds produced in example 3 before and after the fermentation of the corn steep liquor were detected by adopting a headspace solid-phase microextraction method. Specifically, 3 g of unfermented and fermented corn germ meal samples were respectively placed in headspace sampling bottles and heated for 30 min to release volatile substances. SPME extraction heads were inserted for adsorption for 30 min, followed by 15 min of analysis at an injection port of a gas chromatograph. GC conditions: chromatographic capillary column: Equity-5 column (30 mm×0.32 mm×0.25 μm); carrier gas: high-purity helium gas (≥99.999%); flow rate: 2.5 mL/min; temperature of sample injector: 250° C., non-split injection; initial temperature of drying oven: 80° C. maintained for 2 min, increased to 200° C. at a speed of 15° C./min, maintained for 2 min, then increased to 260° C. at a speed of 6° C./min, and maintained at 260° C. for 5 min.

MS conditions: ionization method: electron bombardment source (EI); electron energy: 70 eV; detector voltage: −1499 V; ion source temperature: 250° C.; mass scan range m/z: 15-350. As shown in Table 4, taking example 3 as an example, after treatment with A. niger, compared to the contents of substances before fermentation, the contents of substances that contribute significantly to flavor, such as alcohols, acids, and esters, in the corn steep liquor are improved. In addition, using a digital viscometer from Shanghai Yixin, it is found through viscosity measurement that the viscosity before fermentation (example 3) is 12.51 mPa·s, and the viscosity after fermentation is 1.38 mPa·s.

TABLE 4
Classification and proportions of volatile compounds produced
before and after fermentation of corn steep liquor

| | Percentage (%) of | Percentage (%) of |
|---|---|---|
| | compounds after | compounds before |
| Type of compounds | fermentation (example 3) | fermentation |
| Alcohols | 12.31 | 9.17 |
| Aldehydes | 3.56 | 4.52 |
| Ketones | 6.72 | 8.63 |
| Acids | 7.38 | 5.39 |
| Esters | 9.22 | 7.21 |
| Heterocycles | 5.11 | 3.68 |
| Sulfur-containing | 0.25 | 2.26 |
| compounds | | |
| Others | 55.45 | 59.14 |

The specific examples are only intended to explain the disclosure, instead of limiting the disclosure. After reading this description, those skilled in the art may make modifications to the examples as needed without contributing any inventive labor, which, however, as long as they are within the scope of the claims of the disclosure, still fall within the scope protected by the Patent Law.

What is claimed is:

1. A method for preparing corn steep liquor powder with reduced content of aflatoxins, the method comprises using corn steep liquor as a fermentation raw material and performing fermentation by using Aspergillus niger; and the aflatoxins comprise at least two of aflatoxins Aflatoxin B1 (AFB1), Aflatoxin B2 (AFB2), Aflatoxin G1 (AFG1), and Aflatoxin G2 (AFG2).

2. The method according to claim 1, wherein the A. niger comprises A. niger CCTCC NO: M 2013703.

3. The method according to claim 1, wherein the aflatoxins comprise aflatoxins AFB1, AFB2, AFG1, and AFG2.

4. The method according to claim 1, wherein the method comprises the following steps:
(1) preparation of seed culture solution: preparing A. niger spore suspension, inoculating the A. niger spore suspension into a culture medium, and performing culture to obtain a seed culture solution;
(2) fermentation: inoculating the seed culture solution obtained in step (1) into the corn steep liquor, and performing fermentation to obtain a fermentation product; and
(3) preparation of corn steep liquor powder: collecting the fermentation product obtained in step (2) and performing drying to obtain the corn steep liquor powder.

5. The method according to claim 4, wherein a concentration of A. niger spores in the A. niger spore suspension is not less than 1×10⁶ CFU/mL.

6. The method according to claim 4, wherein step (1) comprises inoculating the A. niger spore suspension into the culture medium according to an inoculation rate of 1%-3%, and performing culture at 25° C.-35° C. for at least 24 hours to obtain the seed culture solution.

7. The method according to claim 4, wherein step (2) comprises inoculating the seed culture solution obtained in step (1) into corn steep liquor according to an inoculation rate of 5%-25%, and performing fermentation at 25° C.-32° C. for 48-72 hours to obtain the fermentation product.

8. The method according to claim 4, wherein step (3) comprises drying the fermentation product obtained in step (2) at 90° C.-105° C. for 24 h-48 hours, and performing crushing.

9. The method according to claim 4, wherein the method comprises the following steps:
(1) preparation of seed culture solution: inoculating the A. niger spore suspension into a culture medium according an inoculation rate of 2%, and performing culture at 30° C. for at least 16 hours to obtain seed culture solution;
(2) fermentation: inoculating the seed culture solution obtained in step (1) into corn steep liquor according to an inoculation rate of 15%, and performing fermentation at 30° C. for 60 hours to obtain a fermentation product; and
(3) preparation of corn steep liquor powder: drying the fermentation product obtained in step (2) at 100° C. for 32 hours, and performing crushing.

10. Corn steep liquor powder prepared by adopting the method according to claim 1.
